(19) United States
(12) Patent Application Publication
(10) Pub. No.: US 2020/0141904 A1
(43) Pub. Date: May 7, 2020

(54) BIOACTIVE COATING FOR SURFACE ACOUSTIC WAVE SENSOR

(71) Applicant: AVIANA MOLECULAR TECHNOLOGIES, LLC, Orlando, FL (US)

(72) Inventors: Soumen Das, Orlando, FL (US); John Martin Hamlyn, Columbia, MD (US); Amitava Gupta, Roanoke, VA (US)

(73) Assignee: AVIANA MOLECULAR TECHNOLOGIES, LLC, Orlando, FL (US)

(21) Appl. No.: 16/629,309

(22) PCT Filed: Jul. 5, 2018
(86) PCT No.: PCT/US18/40887

§ 371 Date: Jan. 7, 2020

Related U.S. Application Data
(60) Provisional application No. 62/530,735, filed on Jul. 10, 2017.
(60) Provisional application No. 62/529,986, filed on Jul. 7, 2017.

(51) Int. Cl. G01N 29/02; G01N 33/553

(52) U.S. Cl. CPC G01N 29/022; G01N 33/553; G01N 2291/0423; G01N 2291/0426; G01N 2291/0255

(57) ABSTRACT

An acoustic wave biosensor component is provided. The acoustic wave biosensor comprising a piezoelectric substrate with or without 3D matrix microstructure to increase the surface of the effective sensing area and an anchor substance covalently bound to a surface of the piezoelectric substrate, and the anchor substance can bind to a capture reagent. A process for fabricating the 3D biosensor surface and component coating the surface of a piezoelectric material with bioactive film comprising an anchor substance is also provided.

### CROSS REFERENCE TO RELATED APPLICATIONS

This patent application claims benefit and priority to U.S. Provisional Patent Application No. 62/529,986 filed on Jul. 7, 2017 and U.S. Provisional Patent Application No. 62/530,735 filed on Jul. 10, 2017, each of which is hereby incorporated by reference in its entirety.

### FIELD

The disclosure relates generally to a bioactive coating method and 3D modification of single or multiplexed biosensor devices with microfluidics using piezoelectric surface of the surface acoustic wave technology. More particularly, the disclosure relates to methods of bio-coating on the surface of a piezoelectric crystal or a metal, to create a three dimensional (3D) surface to increase density of the capturing-agent binding and improve sensitivity implementing sandwich assay for rapid detection of small molecule, nucleic acid sequence, protein, antibody and cell in buffer, biological samples of potentially infected patients or animals, and creates a platform technology suitable for the development of surface acoustic based of biosensors.

### BACKGROUND

Without diagnostics medicine is blind, therefore fast and accurate identification of disease and threat is key in the diagnostic area. Detection technologies used to diagnose biological phenomenon have traditionally employed light and chemical sensors, recent development in acoustic technologies has led to the potential use of acoustic methods for bio-sensing. Acoustic methods utilize the function of a responsive piezoelectric material that responds to an electrical signal with the creation of an acoustic wave (i.e., very high frequency sound) as the fundamental sensing property. As an acoustic wave propagates through or on the surface of the acoustic wave sensor material, and binding of analyte introduce mass loading and/or viscosity changes on the wave path may affect the velocity and/or amplitude of the acoustic surface or bulk waves. These changes may be correlated to the corresponding quantities bound on their surface and are being measured to provide sensing/detection of the said analytes. Unfortunately, the binding between the target molecule and the sensor surface may be weak, therefore the acoustic wave sensors often lack sensitivity and do not operate efficiently when they are presented with the target. As such, there is a need for stable, high intensity immobilization of receptor molecule that will allow the biomolecule/analyte of interest to efficiently bind on the surface to enhance detection sensitivity.

### SUMMARY

In one aspect, the disclosure provides a biosensor component that includes: a substrate coated with a metal; and an anchor substance comprising a binding protein and a functional group having at least one sulfur atom, where the anchor substance binds directly to the metal through the functional group and forms a monolayer on the metal coated substrate; and where the anchor substance is configured to couple to a capture reagent.

In an embodiment, the metal is selected from the group consisting of aluminum, gold, and aluminum-alloy any combination thereof.

In an embodiment, the metal is aluminum.

In an embodiment, the functional group is a thiol group.

In an embodiment, the binding protein is avidin, oligonucleotide, antibody, affimer, aptamer, or polynucleotide.

In an embodiment, the binding protein is avidin selected from the group consisting of neutravidin, natural avidin, strepavidin, and any combination thereof.

In an embodiment, the capture reagent comprises a biotin moiety for binding to the binding protein of the anchor substance.

In an embodiment, the capture reagent comprises a moiety for binding to whole cells, bacteria, eukaryotic cell, tumor cell, virus, fungus, parasite, spore, nucleic acid, small molecules or protein.

In an embodiment, the moiety is selected from the group consisting of antibody, affimer, or aptamer.

In an embodiment, the biosensor further includes an acoustic wave transducer.

In an embodiment, the acoustic wave transducer generates bulk acoustic waves.

In an embodiment, the bulk acoustic wave is selected from the group consisting of thickness shear mode, acoustic plate mode, and horizontal plate mode.

In an embodiment, the biosensor component is a film bulk acoustic-wave resonator-based (FBAR-based) device.

In an embodiment, the acoustic wave transducer generates surface acoustic waves.

In an embodiment, the surface acoustic wave is selected from the group consisting of shear horizontal surface acoustic wave, surface traverse wave, Rayleigh wave, and love wave.

In an embodiment, the substrate comprises a piezoelectric material.

In an embodiment, the metal is coated directed on the substrate.

In an embodiment, the substrate further comprises a dielectric layer and the metal is coated on the dielectric layer.

In one aspect, the disclosure provides a bulk wave resonator including the biosensor component of any one of the foregoing.

In one aspect, the disclosure provides process of coating a surface of a metal material with a bioactive film, including the steps of: applying a first composition comprising an anchor substance to the surface of the metal material to form a monolayer on the surface, where the anchor substance comprises a binding protein and a functional group having at least one sulfur; and applying a second composition comprising a biotinylated capture reagent to the monolayer of the anchor substance, wherein the biotinylated capture reagent binds to the anchor substance through the binding protein to form a layer of the biotinylated capture reagent.

In an embodiment, the surface of the anchor substance to a plasma cleaning.

In one aspect, the disclosure provides a biosensor component including: a piezoelectric substrate; an anchor substance bound to a surface of the piezoelectric substrate, wherein the anchor substance comprises a spacer and a binding component, and a capture reagent, wherein the anchor substance is coupled with the capture reagent thorough the binding component.

In an embodiment, the binding component is a binding protein.

In an embodiment, the binding protein is avidin, oligonucleotide, antibody, affimer, aptamer, or polynucleotide.

In an embodiment, the binding protein is avidin selected from the group consisting of neutravidin, natural avidin, strepavidin, and any combination thereof.

In an embodiment, the binding component is a binding compound having one or more functional group.

In an embodiment, the binding compound has one or more functional group selected from the group consisting of N-Hydroxysuccinimide (NHS), sulfo-NHS, epoxy, carboxylic acid, carbonyl, Maleimide and amine.

In an embodiment, the spacer is a polymer linker.

In an embodiment, the polymer linker IS a polyethylene glycol, polyvinyl alcohol, or polyacrylates.

In an embodiment, the polymer linker IS a polyethylene glycol.

In an embodiment, the anchor substance forms a layer on the surface of the piezoelectric substrate.

In an embodiment, the monolayer the anchor substance forms a self-assembled monolayer on the surface of the piezoelectric substrate

In an embodiment, the binding protein of the anchor substance is extended away from the surface of the piezoelectric substance through the spacer.

In an embodiment, the piezoelectric substrate is selected from the group consisting of quartz lithium niobate and tantalate, 36° Y quartz, 36° YX lithium tantalate, langasite, langatate, langanite, lead zirconate titanate, cadmium sulfide, berlinite, lithium iodate, lithium tetraborate, bismuth germanium oxide, Zinc oxide, aluminium nitride, and gallium nitride.

In an embodiment, the biosensor component further includes a housing and a fluidics chamber wherein the surface of the piezoelectric material bearing the anchor layer forma a wall of the chamber.

In an embodiment, the anchor substance binds to the surface of the piezoelectric substrate through a silane group.

In an embodiment, the binding protein is avidin, oligonucleotide, antibody, affimer, aptamer, or polynucleotide.

In an embodiment, the binding protein is avidin selected from the group consisting of neutravidin, natural avidin, strepavidin, and any combination thereof.

In an embodiment, the biosensor component further includes a capture reagent, wherein the capture reagent comprises a biotin moiety for binding to the binding protein of the anchor substance.

In an embodiment, the capture reagent comprises a third moiety for binding to whole cells, bacteria, eukaryotic cell, tumor cell, virus, fungus, parasite, spore, nucleic acid, protein or small molecules.

In an embodiment, the biosensor component further includes an acoustic wave transducer.

In an embodiment, the acoustic wave transducer generates bulk acoustic waves.

In an embodiment, the bulk acoustic wave is selected from the group consisting of thickness shear mode, acoustic plate mode, and horizontal plate mode.

In an embodiment, the biosensor component is a film bulk acoustic-wave resonator-based (FBAR-based) device.

In an embodiment, the acoustic wave transducer generates surface acoustic waves.

In an embodiment, the surface acoustic wave is selected from the group consisting of shear horizontal surface acoustic wave, surface traverse wave, Rayleigh wave, and love wave.

In one aspect, the disclosure provides a bulk wave resonator comprising the biosensor component of any one of the foregoing.

In one aspect, the disclosure provides a process of coating a surface of a piezoelectrical material with a biofilm, including the steps of: applying a first composition comprising an anchor substance to the surface of a substrate coated with a metal to form a mono layer on the surface, wherein the anchor substance comprises a spacer coupled to a binding component; applying a second composition comprising biotinylated capture reagent to the monolayer of the anchor substance, wherein the biotinylated capture reagent binds to the anchor substance through the binding component of the anchor substance to form a layer of the biotinylated capture reagent.

In one aspect, the disclosure provides a method for determining the presence or quantity of an analyte in a sample the method including the steps of: contacting the biosensor component of any one of the foregoing with a sample; generating an acoustic wave across the coated substrate; and measuring any change in amplitude, phase or frequency of the acoustic wave as a result of analyte binding to the capture reagent.

In one aspect, the disclosure provides a biosensor component including: a piezoelectric substrate; and a capturing reagent immobilized on the piezoelectric substrate, wherein the piezoelectric substrate comprises a three-dimensional (3D) matrix microstructure configured to increase the number of capturing reagent immobilized on the piezoelectric substrate, and wherein the capturing reagent immobilized on the piezoelectric substrate through binding to the 3D matrix microstructure.

In an embodiment, the 3D matrix microstructure comprises a plurality of holes.

In an embodiment, the 3D matrix microstructure comprises a microarray of the capturing agents.

In an embodiment, the 3D matrix microstructure comprises a hydrogel matrix.

In an embodiment, the hydrogel matrix comprises a plurality of holes.

In an embodiment, the hydrogel matrix comprises a cross-linked polymer.

In an embodiment, the cross-linked polymer is hydrophilic.

In an embodiment, the 3D matrix microstructure comprises a dendrimer.

In an embodiment, the 3D matrix microstructure comprises a microarray of the hydrogen matrix.

In an embodiment, the 3D matrix microstructure comprises a layer of the hydrogen matrix.

In an embodiment, the hydrogel matrix is impermeable to whole cells, bacteria, eukaryotic cell, tumor cell, virus, fungus, parasite, spore, nucleic acid, small organic molecule, polypeptide, or protein.

In an embodiment, the biosensor component further includes an anchor substance attaching the capture reagent to the 3D matrix microstructure or the piezoelectric substance.

In an embodiment, the capture reagent comprises a biotin moiety for binding to the binding protein of the anchor substance.

In an embodiment, the capture reagent comprises a moiety for binding to whole cells, bacteria, eukaryotic cell, tumor cell, virus, fungus, parasite, spore, nucleic acid, small organic molecule, polypeptide, or protein.

In an embodiment, the moiety is selected from the group consisting of antibody, affimer, or aptamer.

In an embodiment, the biosensor component further includes an anchor substance.

In an embodiment, the acoustic wave transducer generates bulk acoustic waves.

In an embodiment, the bulk acoustic wave is selected from the group consisting of thickness shear mode, acoustic plate mode, and horizontal plate mode.

In an embodiment, the biosensor component is a film bulk acoustic-wave resonator-based (FBAR-based) device.

In an embodiment, the acoustic wave transducer generates surface acoustic waves.

In an embodiment, the surface acoustic wave is selected from the group consisting of shear horizontal surface acoustic wave, surface traverse wave, Rayleigh wave, and love wave.

In one aspect, the disclosure provides bulk wave resonator including the biosensor component of any one of the foregoing.

In one aspect, the disclosure provides a method of fabricating a biosensor component, comprising: forming a 3D matrix microstructure on a piezoelectric substrate to increase the surface area of the piezoelectric substrate; and immobilizing one or more capturing reagent on the piezoelectric substrate.

In an embodiment, the disclosure includes forming holes on the piezoelectric substrate.

In an embodiment, the method includes forming a hydrogel matrix on the piezoelectric substrate.

In an embodiment, the method includes forming a microarray of hydrogel matrix on the piezoelectric substrate.

In an embodiment, the method includes forming a layer of hydrogel matrix on the piezoelectric substrate.

In an embodiment, the hydrogel matrix comprises a plurality of holes.

In an embodiment, the method includes forming a microarray of the capturing reagent on the piezoelectric substrate using a lithographic printing.

In an embodiment, the method includes forming a layer of dendrimer on the piezoelectric substrate.

In one aspect, the disclosure provides a method for determining the presence or quantity of an analyte in a sample including the steps of: contacting the biosensor component of any one of the foregoing with a sample; generating an acoustic wave across the metal substrate; and measuring any change in amplitude, phase or frequency of the acoustic wave as sample, a result of analyte binding to the capture reagent.

In an embodiment, the sample is environmental or biological

In an embodiment, the biological sample is blood, serum, plasma, urine, sputum or fecal matter.

In an embodiment, the acoustics wave has an input frequency of about 100 to 3000 MHz.

Additionally, some embodiments relate to a biosensor component comprising a substrate coated with a metal, an anchor substance comprising a binding protein or nucleotide and a functional group having at least one sulfur atom, wherein the anchor substance is configured to couple to a capture reagent and binds directly to the metal through the functional group and forms a monolayer on the metal substance.

Some embodiments relate to a process of coating a surface of a metal material/and/or plain crystal surface with a bioactive film by applying a first composition comprising an anchor substance to the surface of the metal/crystal material to form a monolayer on the surface, wherein the anchor substance comprises a binding protein and a functional group having at least one sulfur. A second composition comprises a biotinylated capture reagent to the monolayer of the anchor substance, wherein the biotinylated capture reagent binds to the anchor substance through the binding protein to form a layer of the biotinylated capture reagent.

Some embodiments relate to a biosensor component, a piezoelectric substrate, an anchor substance bound to a surface of the piezoelectric substrate, wherein the anchor substance comprises a spacer and a binding component and a capture reagent, wherein the anchor substance is coupled with the capture reagent thorough the binding component.

Some embodiments relate to a process of coating a surface of a piezoelectric material with a biofilm by applying a first composition comprising an anchor substance to the surface of the metal/crystal material to form a mono layer on the surface, wherein the anchor substance comprises a spacer coupled to a binding component. A second composition comprises a biotinylated capture reagent to the monolayer of the anchor substance, wherein the biotinylated capture reagent binds to the anchor substance through the binding component of the anchor substance to form a layer of the biotinylated capture reagent.

Some embodiments relate to a method for determining the presence or quantity of an analyte in a sample the method which comprises contacting the biosensor component described herein with a sample generating an acoustic or bulk wave across the coated substrate and measuring any change in amplitude, phase or frequency of the acoustic or bulk wave as a result of analyte binding to the capture reagent.

Some embodiments relate to a bulk wave resonator comprising the biosensor component described herein. A piezoelectric substrate with an anchor substance bound to a surface of the piezoelectric substrate, wherein the anchor substance comprises a spacer, a binding component and a capture reagent, wherein the anchor substance is coupled with the capture reagent thorough the binding component.

Some embodiments relate to process of coating a surface of a piezoelectric material with a bioactive coating by applying a first composition comprising an anchor substance to the surface of the metal/crystal material to form a mono layer on the surface, wherein the anchor substance comprises a spacer coupled to a binding component. A second composition comprises a biotinylated capture reagent to the monolayer of the anchor substance, wherein the biotinylated capture reagent binds to the anchor substance through the binding component of the anchor substance to form a layer of the biotinylated capture reagent.

Some embodiments relate to a method for determining the presence or quantity of an analyte in a sample. This method comprises contacting the biosensor component by generating an acoustic or bulk wave across the coated substrate and measuring any change in amplitude, phase or frequency of the acoustic or bulk wave as a result of an analyte binding to the capture reagent.

Some embodiments relate to a biosensor component which comprises a piezoelectric substrate and capturing a reagent immobilized on the piezoelectric substrate, wherein the piezoelectric substrate comprises a three-dimensional (3D) matrix microstructure configured to increase the number of capturing reagent immobilized on the piezoelectric substrate, and wherein the capturing reagent immobilized on the piezoelectric substrate through binding to the 3D matrix microstructure.

Some embodiments relate to a method of fabricating a biosensor component by forming a 3D matrix microstructure on a piezoelectric substrate to increase the surface area of the piezoelectric substrate and immobilizing one or more capturing reagent on the piezoelectric substrate.

Some embodiments relate to a method for determining the presence or quantity of an analyte in a sample the method by contacting the biosensor component of any one of the above embodiments, generating an acoustic bulk wave across the metal substrate and measuring any change in amplitude, phase or frequency of the acoustic or bulk wave as a result of the analyte binding to the capture reagent.

Some embodiments relate to a bulk wave resonator comprising the biosensor component described herein.

Some embodiment use polymers poly(methyl methacrylate) (PMMA) as a Love wave and plasm etching to create a 3D structure on the surface of the sensor to increase the surface area.

The following terms shall have the meaning ascribed to them below.

“Anchor substance” denotes a coating material that binds both to the piezoelectric substrate (for “direct” binding) metal part of the sensor surface or to an intermediary coating thereon and to a “capture reagent” (as defined below). The term includes avidins, a member of a family of proteins functionally defined by their ability to bind biotins, which serve as their specific binding partners (e.g.), avidin, streptavidin, neutravidin), as well as oligo and polynucleotides and proteins having a specific binding partner which could be used to modify a capture reagent and therefore to cause the capture reagent to bind to the anchor-coated piezoelectric sensor material. Also included are naturally occurring carbohydrate-binding lectins, which bind to carbohydrate groups (e.g., on antibodies and antibody fragments (i.e., Fe fragments) and nucleotide fragments such as aptamers). Generally it is not preferred to use a capture reagent as an anchor because of the risk of changing the conformation or even partially denaturing the capture reagent which would affect accuracy of the test. Oligo and polynucleotides can bind to piezoelectric materials through ionic or dipole sites, either directly or through intermediary silver coating applied (e.g., by ion exchange methods). Their specific binding partners are complementary nucleotide molecules and those can be used to modify capture reagents.

“Capture reagent” means a substance that specifically binds to an analyte in a biological sample, such that it can be used to identify and/or quantitate the analyte by capturing it from the biological sample. The term includes antibodies, aptamers and antibody fragments thereof without limitation. A capture reagent will bind to the anchor substance with or without modification with a linking group which is a specific binding partner for the anchor substance (e.g., biotinylation or complementary nucleic acid). In other words, the capture reagent is or comprises a specific binding partner for the anchor substance and simultaneously recognizes an analyte.

A “small organic molecule” refers to an organic molecule, either naturally occurring or synthetic or recombinant, that has a molecular weight of more than about 10 daltons and less than about 2500 daltons, preferably less than about 2000 daltons, preferably between about 10 to about 1000 daltons, more preferably between about 10 to about 500 daltons.

“Avidins” are proteins derived from egg whites, e.g., from avian reptile and amphibian species, and have been used in many biochemical reactions. The avidin family includes neutravidin, streptavidin and avidin, all proteins functionally defined by their ability to bind biotin with high affinity and specificity. Avidins can also include bacterial avidins such as streptavidin and modified avidins like neutravidin (deglycosylated avidin from Thermo Scientific: www.thermoscientific.com). They are small oligomeric proteins, each comprising four (or two) identical subunits, each subunit bearing a single binding site for biotin. When bound to the surface of the biosensor in the present invention, some sites may be facing the metal coated piezoelectric material surface, and are therefore unavailable for biotin binding. Some other sites are facing away from the piezoelectric material and are available for biotin binding. The binding affinity of avidins to biotin, albeit noncovalent, is so high that it can be considered irreversible. The dissociation constant of avidin (KD) is approximately 10—is M, making it one of the strongest known non-covalent bonds. In its tetrameric form, avidin is estimated to be between 66 to 69 kDa in size. Ten percent of the molecular weight is attributed to carbohydrate content composed of four to five mannose and three N-acetylglucosamine residues. The carbohydrate moieties of avidin contain at least three unique oligosaccharide structural types that are similar in structure and composition.

“Biotin”, also known as d-biotin or Vitamin H, Vitamin B7 and Coenzyme R, is a specific binding partner of avidin. It is commercially available from multiple suppliers, including Sigma-Aldrich.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

### BRIEF DESCRIPTION OF THE DRAWINGS

FIG. 1 illustrates one embodiment of bio-coating on a native aluminum surface using a thiolated biological capture reagent.

FIGS. 2A-2C show the preferential neutravidin (NAv) binding to an Aluminum (Al) surface. FIG. 2A shows the results of an enzymatic assay employing a biotinylated HRP/o-Phenylenediamine Dihydrochloride (OPD) pair. The intensity of the absorbance at 417 nm was proportional to the amount of NAv bound to the surface of the sensor. The amount of bound NAv on the Al coated crystal surface was significantly greater when thiolated NAv was used. FIG. 2B illustrates the microscope-based image of biotinylated-fluorescein molecules bound to surface NAv (500× magnification). FIG. 2C shows an image illustrating the binding of 0.2 μm polystyrene biotinylated fluorescent beads (500× magnification).

FIG. 3 illustrates a schematic of the bio-coating development with neutravidin for selectively capturing the target analyte.

FIGS. 4A and 4B illustrate the contact angle measurement of water on the sensor. FIG. 4A shows the plasma cleaning leading to a significant decrease in the contact angle. FIG. 4B shows coating with PEG-silane markedly increased the hydrophobicity of the sensor.

FIGS. 5A and 5B illustrate Fluorescence images of biotinylated fluorescein (FIG. 2C, 50× magnification) and fluorescent polystyrene beads (FIG. 2B, 500× magnification), which show homogeneous binding to the surface bio-coating.

FIG. 6 illustrates the bio-coating development (without neutravidin) for selectively capturing the target analyte.

FIGS. 7A and 7B show a fluorescent analyte that was bound to a surface bio-coating immobilized via an epoxy spacer. FIG. 7A is control (500×) and FIG. 7B is the epoxy coated sensor (500×).

FIGS. 8A and 8B show the SEM image and contact angle of sinusoidal structures in a hydrogel matrix drilled by a picosecond laser system. FIG. 8A shows the sinusoidal structure with periodicity of 25 μm and height of 12 μm. FIG. 8B shows the sinusoidal structure with periodicity of 35 μm and height of 45 μm.

FIG. 9 illustrates the soft lithographic processes for fabrication of micro/nanopattems.

### DETAILED DESCRIPTION

The present disclosure is based, at least in part, on the discovery that a biosensor substrate may be modified before coating or coated with a metal and an anchor substance having a binding protein (e.g., polypeptides, proteins, protein complexes, and the like) including a functional group having at least one sulfur atom, may be bound to the metal coated substrate to form a bioactive coating that may be bound/conjugated to a biosensor device (e.g. a Sound Acoustic Wave (SAW) sensor, Bulk Acoustic Wave (BAW) sensor, and the like) to increase the strength and sensitivity of a signal to be detected by the biosensor device.

In some embodiments, the direct binding of anchor substances, such as avidins, onto a metal coated piezoelectric material, can be obtained under the conditions discussed herein. Using this process, anchor substances are successfully attached directly to a metal coated piezoelectric substrate surface through a strong and stable covalent or chemisorption bond, and form a monolayer on the metal coated piezoelectric surface. The monolayer may contribute to optimal and consistent functioning of the biosensor, as multiple layers of the anchor substance may interfere with the acoustic signal.

Acoustic technologies described herein allow for the use of acoustic methods for biological sensing with high accuracy and sensitivity. The technologies described herein can be used to accommodate and bind biologically sensitive agents onto the surface of the acoustically transmissive materials, which helps further expand the use of acoustic methods for detection application. Some embodiments relate to the use of chemical agents such as silanes, compounds with reactive amine, carboxyl and epoxy residues as well as carbohydrate based materials to provide strong adhesion between the biological materials and the surface of the metal coated crystals. In some embodiments, the crystal surface may be quartz and similar materials such as lithium niobate and tantalate, 36° Y quartz, 36° YX lithium tantalate, langasite, langatate, langanite, lead zirconate titanate, cadmium sulfide, berlinite, lithium iodate, lithium tetraborate, bismuth germanium oxide, Zinc oxide, aluminium nitride, and gallium nitride.

Some embodiments relate to a method of coating the surface of the crystal with a metal that is amenable to the attachment of biomaterials or chemical compounds. In some embodiments, the metal can be aluminum, aluminum alloy, gold, silver, titanium, chromium, platinum, tungsten, etc. In some embodiments, the metal can be aluminum or aluminum alloy. The methods described herein can allow the use of some metal surfaces that may traditionally have poor binding with biomaterials. For example, aluminum by itself may form a weak binding with the biomaterial, but the methods and materials described herein allow a wide application of aluminum surface in a Surface Acoustic Wave (SAW) sensor. The use of aluminum as a surface to bind biomaterial may have advantages such as not causing signal loss, not destroying the binding material, and not forming black or purple plagues when used in conjunction with an acoustic sensor. In addition, aluminum surfaces may propagate acoustic waves more effectively. The methods described herein can help achieve strong binding between biomolecules or chemical molecules and the metal (aluminum or aluminum alloy) coated surfaces and allow the use of metal coated surface in SAW sensors.

The methods and materials described herein can provide stable, covalently bound bio-coatings on the metal (aluminum or aluminum alloy) coated crystal surface, and on the uncoated crystal, these surface bound bioactive coatings can retain functional biological activity. In addition, the methods and materials described herein may help provide a biosensor with high sensitivity when combined with sensitive electrical systems and a variety of modifications.

The methods described herein can achieve covalently bound affinity capture reagents. Some examples of the capture reagents can include but are not limited to small molecules, antibodies, protein antigens, aptamers or other such molecules suitable for the selective capture of a target analyte. In some embodiments, the surface adhesion results in the proper orientation of the said affinity agents on the aluminum surface to selectively and specifically capture the target analyte. In some embodiments, the materials described herein can include the combination of a silane activated with either a thiol functional group used to anchor the affinity agent and affinity agents that are covalently bound to an activated moiety, and the activated moiety can include epoxy and other suitable adhering chemical functional groups. In some embodiments, the activated moieties may be used with spacers such as pegylated carbohydrates for minimum steric hindrance and increase the signal response. In some embodiments, the activated moieties may not be used with spacers.

Biological anchor substances described herein are often known to be bioactive and include, but are not limited to, agents such as avidins. Avidins can bind to a wide range of biotinylated materials including modified proteins, polymers and carbohydrate entities while adding to the stability of the binding. Methods described herein can be used to activate the surface of the SAW sensors, including but not limited to, heat, plasma, radiation and gases such as oxygen or nitrogen. These different processes offer a range of treatments under multiple conditions. The aluminum coated crystal surfaces of the SAW sensors could be activated under these conditions resulting in the enhanced covalent binding of biologically active capture reagents. The combination of these surface modifications and materials serve as a universal platform to decorate the surface of sensors with any antibody or other affinity capture agents for the specific capture of desired target analyte molecules.

### Biosensor Component

The surface of the sensor can be a metal layer (aluminum or aluminum alloy) deposited on a piezoelectric crystal material or the sensor can be an uncoated piezoelectric material with no metal layer. In some embodiments, the surface of the SAW sensor can be a metal layer (aluminum or aluminum alloy), deposited on a piezoelectric crystal material. In some embodiments, sections of the SAW sensor may contain the metal coating, alternating with crystal or may be covered with a dielectric material layer. In some embodiments, the dielectric layer can be a polymer or ceramic layer. In some embodiments, the dielectric layer can comprise SiO₂, poly(methyl methacrylate) (PMMA), zinc oxide, or aluminum nitrogen. In some embodiments, suitable crystals can be used along with various crystal cuts. In some embodiments, sections of the sensor may include a dielectric layer deposited on the piezoelectric substrate. In some embodiments, sections of the sensor may include a dielectric layer deposited on a metal layer, which in turn is deposited on the piezoelectric substrate. In some embodiments, sections of the sensor may include a metal layer deposited on a dielectric layer, which in turn is deposited on a metal layer. In some embodiments, sections of the sensor may include a first metal layer deposited on a dielectric layer, which is then deposited on a second metal layer, the second metal layer is then deposited on the piezoelectric substrate. All suitable approaches regarding the use of sensors for the detection of target analytes can be based on the ability to decorate the sensor surface with a suitable coating described herein. For the detection of biomolecules, the sensor surface can be immobilized or modified with a suitable material that can selectively capture the desired target analyte. In some embodiments, the sensor described herein can be a SAW sensor. In some embodiments, the sensor described herein can be a BAW sensor.

Some embodiments relate to a biosensor component comprising a substrate coated with a metal layer, an anchor substance comprising a binding protein and a functional group having at least one thiol group, wherein the anchor substance binds directly to the metal layer through the functional group and wherein the anchor substance is configured to couple to a capture reagent. In some embodiments, the anchor substance forms a monolayer on the metal layer after binding to the metal layer.

In some embodiments, the metal is selected from the group consisting of aluminum, gold aluminum alloy, silver, titanium, chromium, platinum, tungsten and or any combination thereof. In some embodiments, the metal is deposited on the piezoelectric or dielectric substrate. In some embodiments, the metal is deposited on the dielectric substrate, which is then deposited on another metal layer.

In some embodiments, the substrate comprises a piezoelectric material. In some embodiments, the substrate further comprises a dielectric layer disposed directly above the piezoelectric material.

In some embodiments, the functional group on the binding protein is a thiol group.

In some embodiments, the binding protein is avidin, oligonucleotide, or polynucleotide. In some embodiments, the binding protein can be affinity agents such as antibodies. In some embodiments, the binding protein is avidin selected from the group consisting of neutravidin, natural avidin, strepavidin, and any combination thereof. In some embodiments, the binding protein can include antibody, affimer, and aptamer.

The capture reagent can be an antibody, aptamer, or other specific ligand or receptor formed from biotinylated oligonucleotides, nucleotides, nucleic acids, proteins, peptides, and antibodies including IgA, IgG, IgM, IgE, enzymes, enzyme co-factors, enzyme inhibitors, membrane receptors, kinases, Protein A, Poly U, Poly A, Poly Lysine receptors, polysaccharides, chelating agents, carbohydrate or sugars.

In some embodiments, the capture reagent may comprise a moiety for binding to whole cells, bacteria, eukaryotic cell, tumor cell, virus, fungus, parasite, spore, nucleic acid, protein or small molecules. In some embodiments, the moiety is selected from the group consisting of antibodies, protein fragments, peptides, polypeptides, affimer, antibody fragments, aptamers or nucleotides. In some embodiments, the moiety is selected from the group consisting of antibodies, affimer, or aptamer.

The capture reagent can be modified with a specific binding partner to the binding protein. In some embodiments, the capture reagent further comprises a biotin moiety for binding to the binding protein of the anchor substance.

Some of the exemplified biosensors and detection methods are illustrated as a surface having an antibody attached as a capture reagent. However, the biosensors is not limited to having antibodies as a capture reagent, and can be adapted to immobilize other capture agents including, but not limited to, protein fragments, affimer, antibody fragments, aptamers or nucleotides on the sensor surface.

The biosensor component described herein further comprises an acoustic or bulk wave transducer. In some embodiments, the acoustic wave transducer generates bulk acoustic waves. In some embodiments, the bulk acoustic wave is selected from the group consisting of thickness shear mode, acoustic plate mode, and horizontal plate mode. In some embodiments, the biosensor component is a film bulk acoustic-wave resonator-based (FBAR-based) device.

In some embodiments, the acoustic wave transducer generates surface acoustic waves. In some embodiments, the surface acoustic wave is selected from the group consisting of shear horizontal surface acoustic waves, surface traverse waves, Rayleigh waves, and Love waves.

Some embodiments relate to a biosensor component comprising a crystal layer, an anchor substance comprising a binding protein and a functional group having at least one thiol group, wherein the anchor substance binds directly to the crystal layer through the functional group and wherein the anchor substance is configured to couple a capture reagent.

For embodiments where binding between the metal surface/material and a functional group (e.g., thiol group) is described, the metal surface/material can be replaced with a crystal material or other piezoelectric material.

### Anchor Substance Containing Spacer

Some embodiments relate to a biosensor component comprising a substrate coated with a metal, an anchor substance bound to the metal, wherein the anchor substance comprises a spacer and a binding component and a capture reagent, wherein the anchor substance is coupled with the capture reagent through the binding component. In some embodiments, the substrate can include a piezoelectric material. In some embodiments, the spacer comprises a silane group, which can form a covalent bond on the metal coating. Thus, in some embodiments, the anchor substance binds to the surface of the metal coated piezoelectric substrate through the silane group.

Some embodiments relate to a biosensor component comprising a crystal material and an anchor substance bound to the crystal material, wherein the anchor substance comprises a spacer and a binding component and a capture reagent. The anchor substance is coupled with the capture reagent through the binding component. In some embodiments, the spacer comprises a silane group. The silane group can form a covalent bond on the crystal material. Thus, in some embodiments, the anchor substance binds to the surface of crystal material through the silane group.

In some embodiments, the binding component is a binding protein such as, for example, avidin, oligonucleotide, or polynucleotide. In some embodiments, the binding protein is avidin selected from the group consisting of neutravidin, natural avidin, strepavidin, and any combination thereof.

In some embodiments, the binding component is a binding compound having one or more functional group selected from the groups consisting of N-Hydroxysuccinimide (NHS), sulfo-NHS, epoxy, carboxylic acid, carbonyl, maleimide and/or amine.

In some embodiments, the spacer is a polymer linker where in the Ipolymer linker is a polyethylene glycol, polyvinyl alcohol, or polyacrylates. In some embodiments, the polymer linker is a linear polyethylene having a molecular weight in the range of about 50-about 10,000, about 100-about 10,000, about 200-about 8000, about 300-about 8000, about 400-about 8000, about 500-about 6000, about 600-about 6000, about 700-about 6000, about 800-about 5000, -about 900-about 5000, about 1000-about 5000, about 500-about 4000, about 600-about 4000, about 700-about 4000, about 800-about 4000, about 900-about 4000, about 1000-about 4000, about 500-about 3000, about 600-about 3000, about 700-about 3000, about 800-about 3000, about 900-about 3000, about 1000-about 3000, about 500-about 2000, about 600-about 2000, about 700-about 2000, about 800-about 2000, about 900-about 5000, or about 1000-about 2000.

In some embodiments, the polymer linker is a linear polyethylene having a molecular weight greater than about 10, greater than about 50, greater than about 100, greater than about 200, greater than about 300, greater than about 400, greater than about 500, greater than about 600, greater than about 700, greater than about 800, greater than about 900, greater than about 1000, greater than about 1200, greater than about 1400, greater than about 1600, greater than about 1800, or greater than about 2000. In some embodiments, the polymer linker is a linear polyethylene having a molecular weight of less than about 500, less than about 600, less than about 700, less than about 800, less than about 900, less than about 1000, less than about 1200, less than about 1400, less than about 1600, less than about 1800, less than about 2000, less than about 2200, less than about 2400, less than about 2600, less than about 2800, less than about 3000, less than about 3500, less than about 4000, less than about 4500, less than about 5000, less than about 5500, less than about 6000, less than about 6500, less than about 7000, less than about 7500, less than about 8000, less than about 8500, less than about 9000, less than about 9500, or less than about 10,000.

For embodiments when the binding compound has one or more functional group (e.g., N-Hydroxysuccinimide (NHS), sulfo-NHS, epoxy, carboxylic acid, carbonyl, maleimide, and/or amine), the length of the spacer can be in the range of about 0.1-50, 0.5-50, 1-50, 1.5-50, 2-50, 2.5-50, 3-50, 4-50, 5-50, 0.1-40, 0.5-40, 1-40, 1.5-40, 2-40, 2.5-40, 3-40, 4-40, 5-40, 0.1-30, 0.5-30, 1-30, 1.5-30, 2-30, 2.5-30, 3-30, 4-30, 5-30, 0.1-20, 0.5-20, 1-20, 1.5-20, 2-20, 2.5-20, 3-20, 4-20, 5-20, 0.1-10, 0.5-10, 1-10, 1.5-10, 2-10, 2.5-10, 3-10, 4-10, 5-10, 0.1-8, 0.5-8, 1-8, 1.5-8, 2-8, 2.5-8, 3-8, 4-8, 5-8, 0.1-5, 0.5-5, 1-5, 1.5-5, 2-5, 2.5-5, 3-5, 4-5, 0.1-3, 0.5-3, 1-3, 1.5-3, 2-3, 2.5-3, 0.1-2.5, 0.5-2.5, 1-2.5, 1.5-2.5, or 2-2.5 nm. In some embodiments, the spacer has a length in the range of greater than 0.05, 0.1, 0.2, 0.3, 0.4, 0.5, 0.6, 0.7, 0.8, 0.9, 1, 1.2, 1.4, 1.6, 1.8, 2, 2.5, 3, 4, 5, 6, 7, 8, 9, 10, 20, 30, 40, 50, 60, 70, 80, 90, or 100 nm. In some embodiments, the spacer has a length in the range of less than 1, 1.5, 2, 2.5, 3, 4, 5, 10, 20, 30, 40, 50, 100, 150, 200, 250, or 500 nm.

In some embodiments, the anchor substance forms a monolayer on the surface of the metal coated piezoelectric material. In some embodiments, the anchor substance forms a self-assembled monolayer on the surface of the metal coated piezoelectric material. In some embodiments, the binding protein of the anchor substance is extended away from the surface of the metal through the spacer.

In some embodiments, the piezoelectric substrate is selected from the group consisting of lithium niobate (LiNbO₃), lithium tantalate (LiTaO₃), silicon dioxide (SiO₂), and borosilicate. In some embodiments, the metal coating may be aluminum or an aluminum alloy.

In some embodiments, the biosensor component described herein further comprises a housing and a fluidics chamber wherein the chamber wall is formed on the surface of the coated piezoelectric substrate bearing the anchor substance and the capture reagent.

### Bulk Acoustic Wave Resonator

Bulk Acoustic Wave (BAW) resonator is a device composed at least of one piezoelectric material sandwiched between two electrodes. The electrodes apply an alternative electric field on the piezoelectric material which creates some stress which generate some BAW wave. Some design add some layer with high and low acoustic impedance to build some Bragg reflector and/or suspend these layers. A BAW resonator can include several layers, piezoelectric substrate (AlN, PZT, Quartz, LiNbO₃, Langasite etc.), electrodes (gold, Aluminum, Copper, etc.), Brag reflector (High or Low acoustic impedance material) layers to catch the analyte (bio active layer, antibodies, antigen, gas sensitive layer, palladium, etc.) or any material which can propagate an acoustic wave. The BAW sensor can be a mix of the various layers described herein. The sensitive layer (layer to catch the analyte) can be in contact directly with the electrodes (A), or can be on the Bragg reflector, or can be on any material which can propagate an acoustic wave.

Some embodiments relate to a BAW resonator comprising the biosensor components described herein. Building a BAW sensor for liquid or gas sensing is based on the principle that anything which goes on the surface of the BAW sensors will change its resonant frequency. By tracking and decoding the resonant frequency (measure or phase frequency), the mass loading and the viscosity of the particles attached to the surface of the sensor can be measured.

### Biocoating Method

Some embodiments relate to a process of coating a surface of a metal material with a bioactive film, comprising applying a first composition comprising an anchor substance to the surface of the metal material to form a monolayer on the surface, wherein the anchor substance comprises a binding protein and a functional group having at least one thiol group; applying a second composition comprising a biotinylated capture reagent to the monolayer of the anchor substance, wherein the biotinylated capture reagent binds to the anchor substance through the binding protein to form a layer of the biotinylated capture reagent.

Some embodiments relate to a process of coating a crystal material with a bioactive film, comprising applying a first composition, comprising an anchor substance to the surface of the crystal material to form a monolayer on the surface, wherein the anchor substance comprises a binding protein and a functional group having at least one thiol group. Applying a second composition comprising a biotinylated capture reagent to the monolayer of the anchor substance, wherein the biotinylated capture reagent binds to the anchor substance through the binding protein to form a layer of the biotinylated capture reagent.

Some embodiments relate to a process of coating an aluminum surface with a bioactive film, comprising applying a first composition, comprising an anchor substance to the aluminum surface to form a monolayer on the aluminum surface, wherein the anchor substance comprises a binding protein and a thiol functional group; and applying a second composition comprising a biotinylated capture reagent to the monolayer of the anchor substance, wherein the biotinylated capture reagent binds to the anchor substance through the binding protein to form a layer of the biotinylated capture reagent. The process can also be used for coating a crystal surface or the surface of a dielectric material.

Some embodiments relate to a process for coating the surface of a metal coated piezoelectric material with a bioactive film comprising treating a surface of the metal coated piezoelectric material to activate the metal surface and applying a layer of the anchor substance directly to the activated surface of the metal coated piezoelectric substrate. The anchor substance has the properties to bind to a capture reagent comprising or constituting a specific binding partner for the anchor substance. In some embodiments, the anchor substance comprises a silane functional group. The silane functional group is capable of reacting with the metal coated piezoelectric surface. In some embodiments, the method further comprises depositing a metal layer on a piezoelectric substrate. In some embodiments, the metal is aluminum. The process can also be used for coating a crystal surface or the surface of a dielectric material.

In some embodiments, the method comprises forming a chemisorbed anchor layer on the metal surface with a covalent bonding.

Some embodiments provide a method for determining the presence or quantity of an analyte in a biological fluid sample the method comprising contacting the biosensor component with a composition comprising a capture reagent, wherein the capture reagent comprises or constitutes a specific binding partner for the anchor substance and also specifically recognizing an analyte. causing the capture reagent to bind to the anchor substance, forming a capture reagent layer, while contacting the bound capture reagent layer with a biological fluid sample and generating an acoustic wave across/through the piezoelectric surface and measuring any change in amplitude, phase, time-delay or frequency of the wave as a result of analyte binding to the capture reagent layer.

Some embodiments relate to a process of coating a surface of a metal material with a bioactive film, comprising applying a first composition, comprising an anchor substance to the surface of the metal material to form a monolayer on the surface, wherein the anchor substance comprises a spacer coupled to a binding component and applying a second composition comprising biotinylated capture reagent to the monolayer of the anchor substance, wherein the biotinylated capture reagent binds to the anchor substance through the binding component of the anchor substance to form a layer of the biotinylated capture reagent.

In some embodiments, the method described herein further includes activating the surface of the anchor substance. In some embodiments, activing the surface of the anchor substance comprises plasma cleaning. In some embodiments, plasma cleaning includes using oxygen or an oxygen/argon mixture to treat the surface. In some embodiments, the plasma cleaning lasts for 1-10 min, 1-20 min, 1-30 min, or 1-60 min. In some embodiments, the plasma cleaning lasts for longer than 1 min, 5 min, 10 min, 20 min, 30 min, 40 min, 50 min, 60 min, 1.5 h, 2 h, 3 h, or 4 h. In some embodiments, the plasma cleaning lasts for less than 5 min, 10 min, 20 min, 30 min, 40 min, 50 min, 60 min, 1.5 h, 2 h, 3 h, or 4 h. In some embodiments, the plasma cleaning includes treatment at 50-200 watts of 50-150 KHz.

In some embodiments, the method described herein is direct coating. In some embodiments, the direct coating involves simple and rapid coating chemistries that are executed in seconds or minutes rather than hours and are manufactured using a scalable, continuous and in-line method such as ink-jet printing with required precision and ability to dispose a monolayer of substance, easily automated with minimal operator intervention. This coating method produces a low number of rejects and generates smaller amounts of hazardous waste. This coating method deposits anchor substances directly on the piezoelectric surface without an intermediary layer of material.

In some embodiments, the preparation method described herein comprises cleaning of the piezoelectric substrate surface. The cleaning step can be accomplished by a number of methods, including, but not limited to, acid treatment, ultraviolet exposure and various methods of plasma treatment which can remove virtually all organic contaminants on the surface of the piezoelectric substrate via the generation of highly reactive species. In some embodiments, the preparation method comprises plasma cleaning.

### Binding of Analytes to the Coated Biosensor

In some embodiments, the bound avidin on the piezoelectric substrate surface requires activation to bind analytes of interest. The activation includes a biotinylated binder such as an antibody, which is specific to an analyte antigen of interest. The antibody or other agent is biotinylated prior to its affixation to the avidin-coated chip. The antibody can bind to its analyte antigen before or after it is affixed to the avidin substrate. The analyte biotinylated antibody complex can be formed outside of the sensor and the complex can be contacted with the sensor, whereby the biotin on the antibody will bind to the avidin-coated chip. Which of the two methods is preferred is dependent upon the analyte and on the sample processing. Both methods are within the scope of the present disclosure. Analysis of the surface coating with a particular antibody bound to avidin on the chip surface resulted in a determination for depths of 6 to 9 nm, again using AFM, demonstrating that antibody is indeed bound to the avidin layer.

Antigen-specific biotinylated capture reagents are applied to form a second layer consisting of bound and excess free biotinylated reagent in a non-drying medium also containing protein stabilizers known in the art such as, sucrose, trehalose, glycerol and the like. Many agents can be biotinylated, the most commonly used amongst them is biotinylated antibodies, specifically recognizing an analyte of interest. Protein capture reagents can be biotinylated chemically or enzymatically. Chemical biotinylation utilizes various known conjugation chemistries to yield nonspecific biotinylation of amines, carboxylates, sulfhydryls and carbohydrates. It is also understood that N-hydroxy succinimide (NHS)-coupling gives biotinylation of any primary amines in the protein. Enzymatic biotinylation results in biotinylation of a specific lysine within a certain sequence by a bacterial biotin ligase. Most chemical biotinylation reagents consist of a reactive group attached via a linker to the valeric acid side chain of biotin. Enzymatic biotinylation is most often carried out by linking the protein of interest at its N-terminus, C-terminus or at an internal loop to a 15 amino acid peptide, termed AviTag or Acceptor Peptide (AP). These biotinylation techniques are known in the art.

Once bound, the capture reagent is briefly exposed to heated air to effect partial removal of water from the applied fluid forming a protective and stabilizing gel that will ensure long-term stability of bound proteinaceous binders like antibodies in a non-drying gel layer which allows essentially complete time-dependent formation of the second antigen-specific binder layer. These glass-like layers are optionally dehydrated for storage in the presence of desiccant pellets of silica or molecular sieves inside the pouch of the cartridge. The upper chamber of the cartridge is sealed to form a fluidic compartment. The cartridge with the upper chamber is then sealed inside a plastic storage pouch, preferably in a N2 atmosphere.

The binding between anchor substance (e.g., avidin) and biotinylated capture reagent causes a second, capture reagent layer to form on the chip. Prior to use, any residual unbound biotinylated capture reagent and other components in the protective gel layer can be readily removed by a simple flush with an assay buffer or even with the specimen fluid during the analytical procedure. These sensors have been demonstrated to detect antigens, binding of Analytes and Disease Detection using the Biosensor.

Biosensors described herein can be used to detect a variety of agents and biochemical markers when outfitted with the appropriate right biofilm coating which contains a capture agent that specifically binds to the analyte of interest. Examples of the uses to which this integrated biosensor can be put include human and veterinary diagnostics. Analyte is defined as any substance that is or that is found in or generated by an infectious agent and that can be used in detection including, without limitation, small molecules, oligonucleotide, nucleic acid, protein, peptide, pathogen fragment, lysed pathogen, and antibodies, including IgA, IgG, IgM, IgE, enzyme, enzyme co-factor, enzyme inhibitor, toxin, membrane receptor, kinase, Protein A, Poly U, Poly A, Poly Lysine, polysaccharides, aptamers, and chelating agents. Detection of antigen-antibody interactions have been previously described (U.S. Pat. Nos. 4,236,893, 4,242,096, and 4,314,821, all of which are expressly incorporated herein by reference). Further, the application in the detection of whole cells (including prokaryotic, such as pathogenic bacteria, eukaryotic cells, and mammalian tumor cells), viruses (including retroviruses, herpes viruses, adenoviruses, lentiviruses, etc.), fungus, parasites and spores, (included phenotypic variations, of infections agents, such as serovars or serotypes) are within the scope of this disclosure.

Some embodiments relate to a method for determining the presence or quantity of an analyte in a sample, the method comprising contacting the biosensor component with the sample; generating an acoustic wave; measuring a change in amplitude, phase or frequency of the acoustic wave as a result of analyte binding to a capture reagent.

In some embodiments, the sample is an environmental or biological sample. In some embodiments, the biological sample is blood, serum, plasma, urine, sputum or fecal matter.

In some embodiments, the substrate coated with metal is a piezoelectric substrate. In some embodiments, the acoustics wave has an input frequency of about 10 to 3000 MHz. In some embodiments, the acoustics wave has an input frequency in the range of about 1-10000, 1-8000, 1-6000, 1-5000, 1-4000, 1-3000, 1-2000, 1-1000, 10-8000, 10-6000, 10-5000, 10-3000, 10-2000, 10-1000, 50-8000, 50-6000, 50-5000, 50-3000, 50-2000, 50-1000, 100-8000, 100-6000, 100-5000, 100-3000, 100-2000, 100-1000, 200-8000, 200-6000, 200-5000, 200-3000, 200-2000, 200-1000 MHz. In some embodiments, the acoustics wave has an input frequency greater than about 1, 5, 10, 20, 30, 40, 50, 60, 70, 80, 90, 100 MHz. In some embodiments, the acoustics wave has an input frequency lower than about 500, 1000, 2000, 3000, 4000, 5000, 6000, 7000, 8000, 9000, 10000 MHz.

Embodiments disclosed herein provide individual elements and sensors which exhibit a combination of the independent advantages found in each of the two sensor classes disclosed above. For example a first embodiment of using an anchor substance having a thiol functional group to bind to the piezoelectric substrate can be combined with the embodiments of the anchor substance having a spacer.

### 3D Surface to Improve the Coting Density of the Anchoring Agent

Many sensor designs utilize a matrix (or a plurality of matrices) such as an enzymatic hydrogel matrix to function. The term “matrix” is used herein according to its art-accepted meaning of something within or from which something else originates, develops, takes form and/or is found. An exemplary enzymatic hydrogel matrix typically comprises a bio-sensing enzymes (e.g. glucose oxidase or lactate oxidase) and human serum albumin proteins that have been cross-linked together with a crosslinking agent such as glutaraldehyde to form a polymer network. This network is then swollen with an aqueous solution to form an enzymatic hydrogel matrix. The degree of swelling of this hydrogel frequently increases over a time-period of several weeks, and is presumably due to the degradation of network cross-links. Regardless of its cause, an observed consequence of this swelling is the protrusion of the hydrogel outside of the hole or “window” cut into the outer sensor tubing. This causes the sensor dimensions to exceed design specifications and has a negative impact on its analytical performance.

Some embodiments relate to utilizing a plurality of different micro patterns to increase the effective surface area of the immobilized capturing reagents bound without compromising the steric configuration of the binding moiety (e.g., antibody) that is effective in conjugating to target analytes (e.g., antigens either free or expressed on the surface of cells or viral particles). Since the surface density of the bound analyte (e.g., antigen) is critical in determining the performance of the sensor, it is necessary to ensure a relatively open three-dimensional matrix microstructure allowing diffusive transport of the target analyte, and also accommodating the size of the binding moiety on the anchor substance (e.g., avidin molecules) to which the capturing reagent (e.g., antibody conjugated biotin) is bound. Some embodiments relate to detect intact pathogen species, including viruses and bacteria, which would require passages of about 0.05 microns-about 10 microns in width through the three-dimensional (3D) matrix in order to allow transport of these species through the matrix comprising capturing reagents (e.g., activated biotin molecules).

Some embodiments relate to biosensor elements having enhanced material properties and biosensors constructed from such elements. The disclosure further provides methods for making and using such sensors. While some embodiments pertain to acoustic wave sensor, a variety of the elements disclosed herein (e.g. piezoelectric substrate and 3D matrix microstructure designs) can be adapted for use with any one of the wide variety of sensors known in the art. The analyte sensor elements, architectures and methods for making and using these elements that are disclosed herein can be used to establish a variety of layered sensor structures. Such sensors exhibit a surprising degree of sensitivity and accuracy. The sensors also have a high degree of flexibility, versatility and characteristics which allow a wide variety of sensor configurations to be designed to examine a wide variety of analyte species.

Compared to traditional SAW sensors, the sensitivity of the biosensors described herein can be sufficient for the detection of biological analytes at a low concentration and also for the detection of bacterial or viral infections where the number of infectious particles in biological fluids may be small. Further, the sensors described herein also have sufficient sensitivity in situations wherein the volumes of biological fluids are also limited.

The detection and quantifying method described herein can have sensitivity sufficient to detect biological analytes in the picomolar range and also for the detection of bacterial or viral infections where the number of infectious particles in biological fluids may be small (i.e., <10 particles/ml). In addition, the detection method described herein can also be used when the volumes of biological fluids are also limited (e.g., 10-250 microliters) due to its enhanced sensitivity.

The 3D matrix microstructure can include a 3D gel or a nanostructured surface, utilize a 3D super-molecular architecture, which can be integrated in the biosensor to increase the effective surface area and the number of capturing agents immobilized on the surface of the biosensor. Various dendrimers can be used to create the 3D matrix microstructure since dendrimers provide a high density of functional groups in 3D space in a branched configuration. The peripheral functional group facilitates the conjugation of antigen/antibody on the sensor surface. Due to their branch structure, dendrimers also reduce steric hindrance of antigen and antibody binding, therefore facilitate the capture of the target molecule. Polyamidoamine (PAMAM) and/or polypropylenimine (PPI) dendrimers can be used to coat the surface of the piezoelectric substrate. Dendrimers can be coated on the surface of the piezoelectric substrate either covalently or non-covalently. Sialinization method can be used to functionalize the piezoelectric surface using aminosilane, cyanosilane, epoxysilane, etc. Dendrimers can be covalently conjugated with the functionalized surface acoustic surface. The height of the dendrimer layer can be between 5-20 nm. Next, the antibody, antibody fragment, single domain antibody, small molecular, DNA or antigen can be immobilized on the peripheral of the dendrimer surface to capture the target molecule.

### Method of Fabricating a Three-Dimensional Matrix Microstructure

Lithographic technologies can be used to form the 3D matrix microstructure on the piezoelectric substrate. Photomasks and molds can be used to form the micro pattern during the photo polymerization process. After the lithographic process, the anchor substance can be attached to the microstructure. After the anchor substance is attached to the 3D matrix microstructure, the capturing reagent can be then immobilized onto the microstructure by coupling to the anchor substance.

Some embodiments relate to a method of forming a 3D matrix microstructure on a piezoelectric substrate, said method comprises applying a suspension to said piezoelectric substrate to form a suspension layer, applying a photomask to said suspension layer, exposing said photomask to an ultraviolet light source, whereby said portions of said suspension layer not covered by said photomask are reacted and removing any unreacted suspension layer from said substrate, wherein said 3D matrix microstructure is formed on said substrate.

Some embodiments relate to a method of forming a 3D matrix microstructure on a piezoelectric substrate, said method comprises forming a microfluidic network comprising at least one microchannel on said piezoelectric substrate, filling said microchannel with a hydrogel precursor solution, exposing said hydrogel precursor to an ultraviolet light source and removing said microfluidic network from said piezoelectric substrate leaving said three-dimensional hydrogel microstructure disposed on said substrate.

In some embodiments, a portion of said suspension layer is not covered by said photomask.

In some embodiments, said suspension is applied to said substrate by spin-coating.

In some embodiments, said suspension is applied to said substrate by flowing in a microfluidic channel.

In some embodiments, said unreacted suspension layer is removed from said substrate by dissolving said suspension layer in a solvent, wherein the solvent can be water, saline, a phosphate buffered saline or an organic solvent. In some embodiments, said unreacted suspension layer is removed from said piezoelectric substrate by washing.

In some embodiments, the method described herein further comprises exposing the 3D matrix microstructure to a solution containing a binding reagent. In some embodiments, the binding reagent is biotin.

In some embodiments, the method described herein further comprises exposing the 3D matrix microstructure to a solution of anchor substance, wherein the 3D matrix microstructure comprises a binding reagent, and wherein the 3D matrix microstructure is attached to the anchor substance through the binding reagent.

In some embodiments, the method described herein further comprises exposing the 3D matrix microstructure to a solution of capturing agents after the anchor substance is attached to the 3D matrix microstructure.

Some embodiments relate to a method of fabricating a biosensor component, comprising forming a 3D matrix microstructure on a piezoelectric substrate to increase the surface area of the piezoelectric substrate and immobilizing one or more capturing reagent on the piezoelectric substrate.

In some embodiments, the fabrication method described herein further comprises forming holes on the piezoelectric substrate.

In some embodiments, the fabrication method described herein further comprises forming a hydrogel matrix on the piezoelectric substrate.

In some embodiments, the fabrication method described herein further comprises forming a microarray of hydrogel matrix on the piezoelectric substrate.

In some embodiments, the fabrication method described herein further comprises forming a layer of hydrogel matrix on the piezoelectric substrate.

In some embodiments, the fabrication method described herein further comprises the hydrogel matrix comprises a plurality of holes.

In some embodiments, the fabrication method described herein further comprises forming a microarray of the capturing reagent on the piezoelectric substrate using a lithographic printing.

In some embodiments, the fabrication method described herein comprises forming holes using a laser. In some embodiments, the laser is a picosecond or femtosecond pulsed laser.

### Example 1

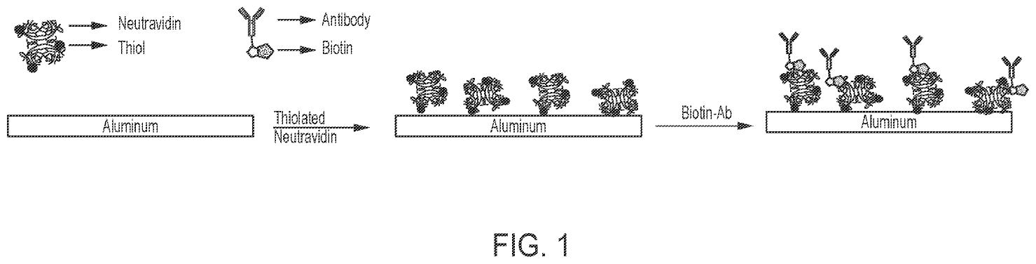

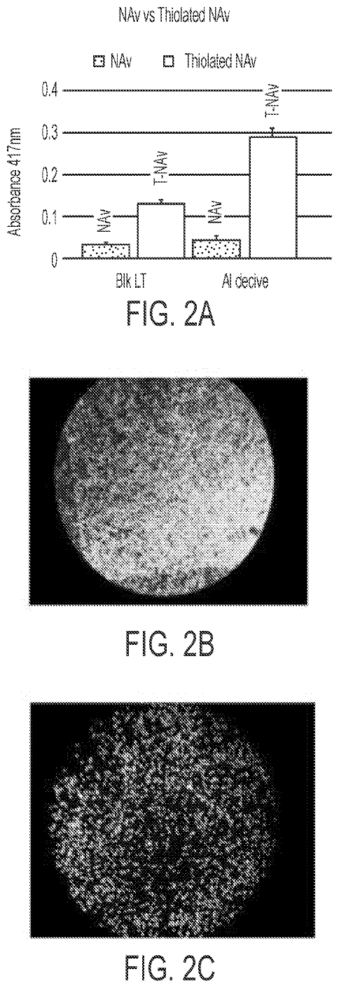

Thiolated-neutravidin was used as a first layer that attached directly to the sensor surface. FIG. 1 illustrates bio-coating of a native aluminum surface using a thiolated biological capture reagent. Aluminum was provided as an example but can also be used on crystal. The attachment was based upon thiol chemistry. Thiols have a high avidity for gold surfaces, forming stable covalent bonds. FIG. 2A shows that thiolated-neutravidin also exhibited high avidity for aluminum with an unanticipated preferential binding for aluminum versus the crystal. The preferential binding of thiolated-neutravidin to the aluminum metal can be used to selectively fabricate any area of interest on the sensor. The capture agent can be thiolated-neutravidin and it can also be replaced by any thiolated capture agent, including aptamers, nucleotides, and antibodies. The method described herein can be used for other materials used for transmission of acoustic waves such as titanium, etc.

Small liquid volumes in the low microliter range containing 10-0.01 mg/ml neutravidin in ddH20 were applied to the target surface of the sensor area and allowed to air dry for a period of time, varying from minutes to hours, based on the condition of the crystal or aluminum surface. Excess unabsorbed neutravidin was subsequently washed away thoroughly. FIG. 2A compares the binding of a biotinylated enzymatic probe for surface coated neutravidin and thiolated-neutravidin, respectively, using an optical assay method. The data shows that the absorption of thiolated-neutravidin to aluminum was approximately six fold greater than to the crystal surface and also greater than the absorption for (non-thiolated) neutravidin.

FIGS. 2A-2C show the preferential neutravidin (NAv) binding to an Aluminum surface. FIG. 2A shows the results of an enzymatic assay employing a biotinylated HRP/o-Phenylenediamine dihydrochloride (OPD) pair. The Blk LT represents the lithium tantalite crystal surface. The intensity of the absorbance at 417 nm was proportional to the amount of neutravidin bound to the surface of the sensor. The amount of bound neutravidin on the aluminum or crystal surface was significantly greater when thiolated neutravidin was used. FIG. 2B illustrates the microscope-based images of biotinylated-fluorescein molecules bound to surface neutravidin (500× magnification), and FIG. 2C illustrates the binding of 0.2 μm polystyrene biotinylated fluorescent beads (500× magnification). The presence of surface-bound fluorescence on the SAW sensors using biotinylated fluorescein and polystyrene fluorescent beads confirmed that the surface neutravidin bio-coating was functionally active.

### Example 2

The aluminum or crystal surface was first activated by plasma cleaning (minutes to hours). The exposure of the sensor surface to plasma cleaning created functional groups that can be readily measured by evaluating the water contact angle. Contact angles significantly less than 90° were optimal for subsequent attachment of reagents to the activated surface. The activated surface was later exposed to thiolated-neutravidin to form a layer on the surface. Following coating, the sensor was washed to remove excess thiolated-neutravidin from the activated aluminum or crystal surface. The coated device was then dried.

### Example 3

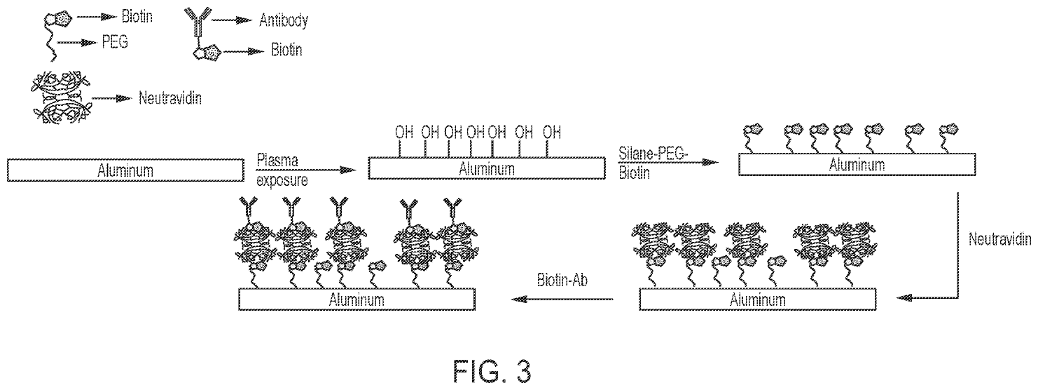

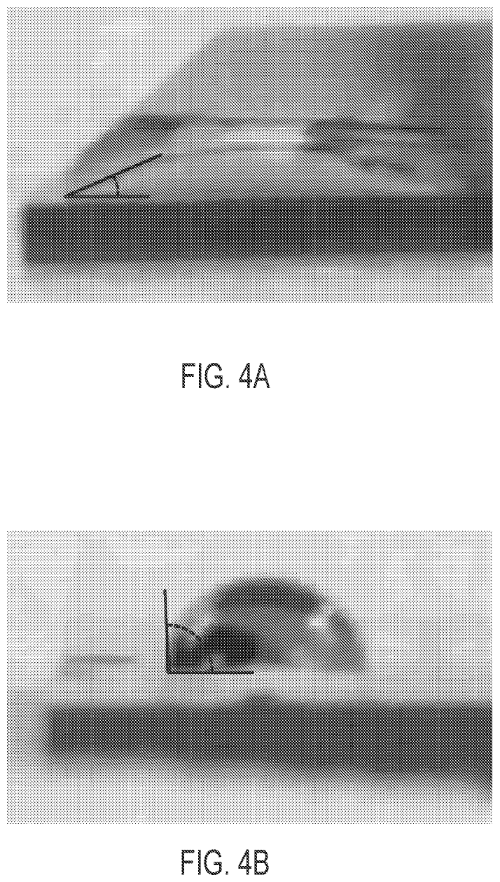

FIG. 3 illustrates a schematic of the bio-coating development with neutravidin for selectively capturing the target analyte. The aluminum or crystal surface was first activated by plasma cleaning (minutes to hours). The exposure of the sensor to plasma cleaning created functional groups that can be readily measured by evaluating the water contact angle. Contact angles significantly less than 90° were optimal for subsequent attachment of reagents to the activated surface. FIGS. 4A and 4B illustrate the contact angle measurement of water on the sensor. FIG. 4A shows the plasma cleaning leading to a significant decrease in the contact angle, and FIG. 4B shows coating with PEG-silane markedly increased the hydrophobicity of the sensor.

The activated surface was subsequently coated with a silane attached to a pegylated compound of various lengths (the spacer) with biotin covalently attached to the top of the spacer. The concentration of the PEG was important to ensure a monolayer and depended on the reaction conditions used. Following the coating, the sensors were washed to remove excess unabsorbed PEG-Biotin from the activated aluminum surface. The coated devices were then dried. The integrity of the PEG-biotin coating was then confirmed with the water contact angle measurement. The length of the PEG spacer can be between 100-2000 molecular weight and can be tailored for a particular binding agent of interest.

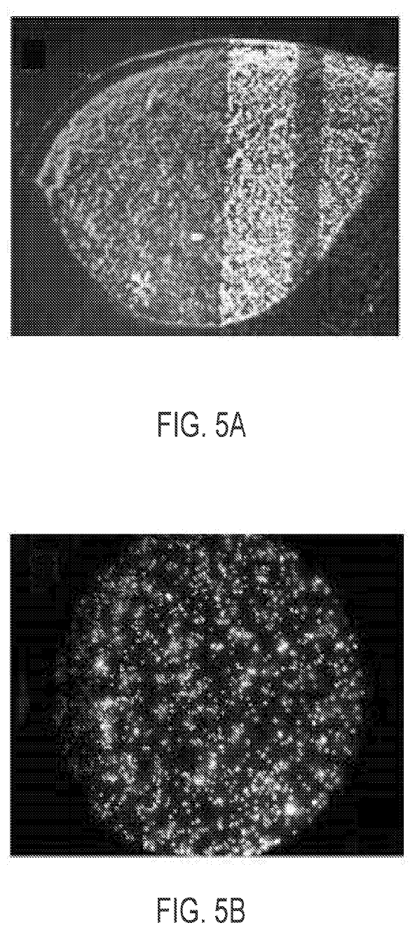

FIGS. 5A and 5B illustrate Fluorescence images of biotinylated fluorescein and fluorescent polystyrene beads. FIG. 5A being biotinylated fluorescein (50× magnification) and FIG. 5B being fluorescence polystyrene beads (500× magnification) showing homogeneous binding to the surface bio-coating. FIGS. 5A and 5B show that biotinylated polystyrene beads adhered to the activated surface of the sensor and demonstrated that the surface neutravidin obtained was fully functional. Similarly, any biotinylated substance including biotinylated antibodies or their fragments, aptamers, etc., can be fabricated on the bio-coating. In the example shown, biotinylated-fluorescein was used as the probe for surface-bound neutravidin. Under conditions where all neutravidin binding sites on the surface were taken, for example, by a biotinylated antibody, the subsequent addition of biotinylated-fluorescein cannot be observed on the bio-coating (data not shown). The data indicates that the coating was suitable for the capture of a target analyte. Accordingly, the binding of an analyte to the surface of the sensor would result in a change in micro-viscosity and mass loading on the sensor surface that can be detected quantitatively using SAW technology.

### Example 4

This example involves a process for the bio-coating of aluminum and/or crystal following surface activation and derivatization. Direct covalent attachment of a biological capture agent (e.g., antibody) to the surface via a short NHS or epoxy spacer.

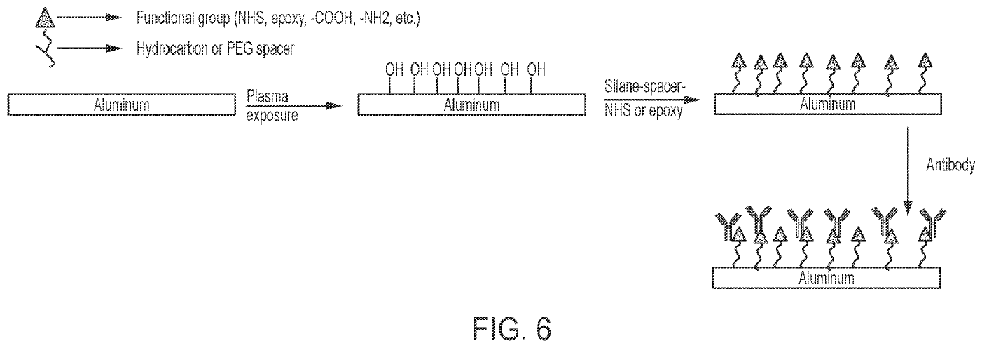

FIG. 6 illustrates the bio-coating development (without neutravidin) for selectively capturing the target analyte. A functional group attached to the spacer (e.g., PEG or carbohydrate chain) was used that can bind directly to an antibody or other analyte capturing molecule attached to the sensor surface. This direct approach avoided the use of neutravidin and thereby reduced the overall thickness of the bimolecular layer and the number of process steps involved. Either N-Hydroxysuccinimide (NHS), sulfo-NHS, maleimide, —COOH or —NH2 or 3-glycidoxypropyl (epoxy) functional group with a short spacer (2 to 20 nm long PEG or carbohydrate chain) can be selected. In each case, silane was used as an anchoring molecule to attach the functional groups separated by a spacer. The functionalized spacer reacted with the capture molecule (e.g., antibody) resulting in high density and reduced steric hindrance. The aluminum or crystal surface of the sensor was activated by plasma cleaning. Next, silane molecules (5-10% concentration-wt/volume) were applied to the sensor surface and incubated (minutes to hours). The excess silane was washed off with solvent. For NHS functionalization, antibody/protein (1-10 ug) was applied directly to the sensor and incubated at room temperature. Following either NHS or epoxy functionalization, a fluorescent-analyte can be added to confirm the functionality of the surface bio-coating.

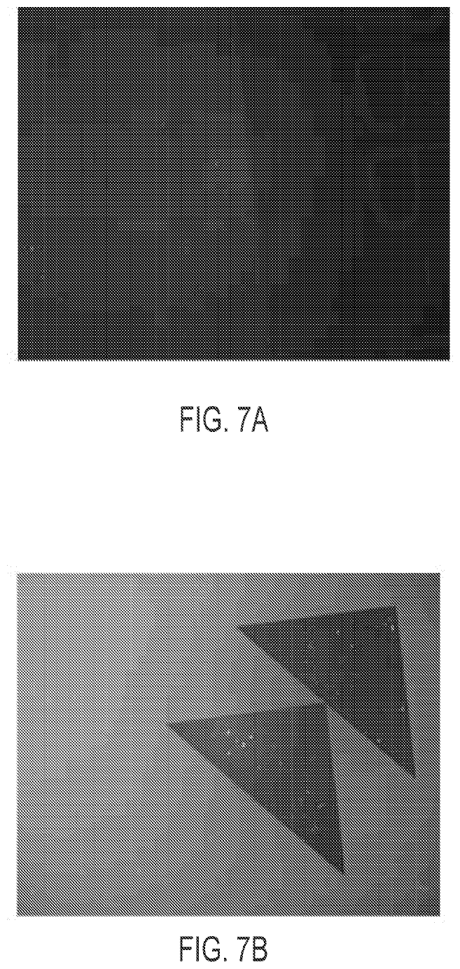

FIGS. 7A and 7B show a fluorescent analyte that was bound to a surface bio-coating immobilized via an epoxy spacer. FIG. 7A is control (500×) and FIG. 7B is the epoxy coated sensor (500×).

### Example 5

A three-dimensional matrix in the piezoelectric substrate can be formed by drilling holes into the surface so as to expose a greater area for capturing agents and this structure can help increase the activated area of the piezoelectric substrate. The holes are preferred to be as deep as possible, since increasing depth of each hole increases the area of avidin exposed to the analyte per hole. However, increasing the depth of the hole may also increase the aspect ratio of the hole, and may make it more difficult for the analyte to diffuse down to the bottom of the hole. An aspect ratio of greater than IO (h/R) is not preferred, because it will unduly increase the time needed for the analyte to cover the entire area of the hole, causing the response time of the instrument. An aspect ratio of IO (h/R) or less is preferred. If the area of the piezoelectric substrate coated with capturing agents is 100 mm² and holes of diameter 20 microns and depth of 100 microns are added to it, the contact area increases by 6280 microns² per hole. Therefore 1.6×10⁴ holes are needed to double the total contact area, and the number of holes needed to increase the contact area by a factor of 103 is 1.6×10′. The surface area occupied by 1.6×10′ holes is 50 sq. mm. The area density of holes is thus 50%, quite achievable by a scanning pulsed picosecond laser. For increases in surface area greater than 103, holes of greater diameter are preferred. For example, the number of holes of diameter 100 microns and depth of 500 microns will double the contact area is 1.3×10² covering a total surface area of 1.04 mm².

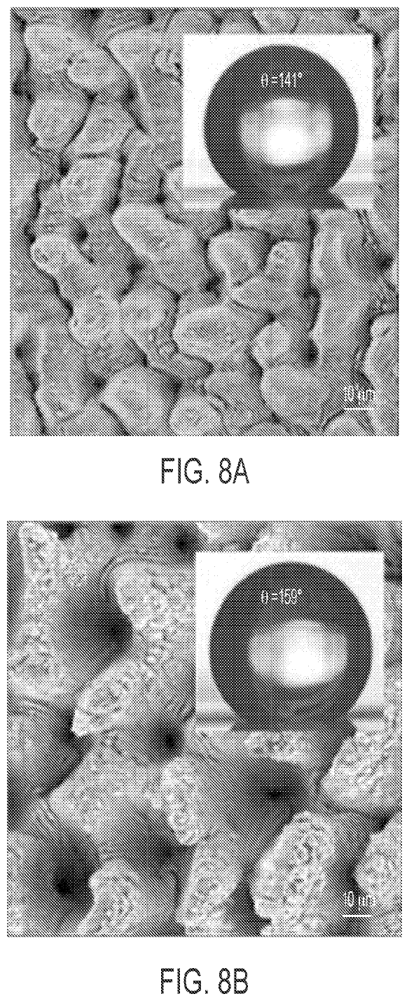

FIGS. 8A and 8B show the SEM image and contact angle of sinusoidal structures in a hydrogel matrix drilled by a picosecond laser system, operating at 1.04μ. FIG. 8A shows the sinusoidal structure with periodicity of 25 μm and height of 12 μm, and FIG. 8B shows the sinusoidal structure with periodicity of 35 μm and height of 45 μm. Holes of these dimensions in a hydrophilic cross-linked matrix are preferably drilled by using a picosecond or femtosecond pulsed laser.

### Example 6

A microarray of biotin streptavidin complex is immobilized on the piezoelectric substrate. Preferably, each dot can have a diameter of 10-25 microns, and can have a height of 5-20 microns. Preferably, a surface packing density of 25-50% can be achieved using conventional protein microarray technology. Protein microarrays are conveniently fabricated using polyethylene glycol (PEG) as a coating material that is highly inert to adsorption of proteins including streptavidin and biotin. Photolithographic techniques are used to pattern the PEG coating before attaching biotin which is then complexed to streptavidin before binding biotin complexed with antibodies.

### Example 7

A hydrogel matrix is formed by polymerization and cross-linking of a hydrophilic monomer formulation incorporating a water soluble inert diluent such as PEG. The hydrogel matrix can be formed by adding a layer of the monomer formulation on a piezoelectric substrate, then exposing the monomer layer to ultraviolet radiation so as to activate the photo initiator therein, and effect polymerization and cross-linking. The hydrogel matrix is then immersed in deionized water or PBS for several minutes to hours, depending on the thickness of the matrix, its hydrophilicity (equilibrium water content), and treatment temperature. This treatment removes the diluent, replacing it with water introducing micro-cavities, and enhancing the free volume that allows local mobility of the segments of protein molecules to be attached to the matrix. The matrix is then eluted with a solution of biotin (i.e., NHS-LC-biotin, dissolved in phosphate buffered saline, in order to bind biotin on the hydrogel matrix. The hydrogel matrix is then further eluted with a solution of streptavidin to bind streptavidin to the bound biotin. The functionalized hydrogel matrix is then ready to be activated with biotin conjugated with antibodies targeting specific pathogens or other bio-agents.

### Example 8

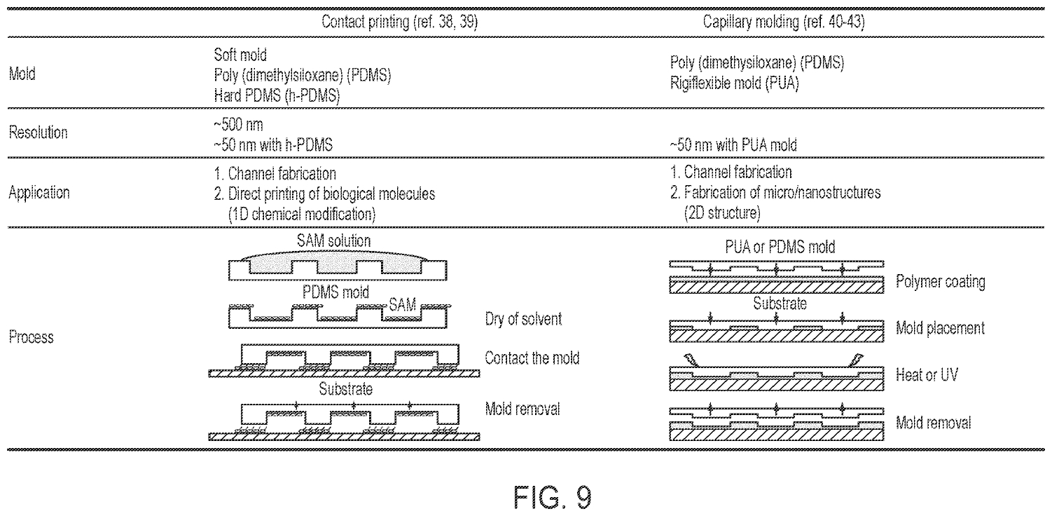

The micro pattern and the 3D matrix described in Examples 5-7 can all be prepared using a soft lithographic process on the piezoelectric substrate. This approach that is especially amenable to automation as shown in FIG. 9. The soft lithographic processes for fabrication of micro/nanopatterns shown in FIG. 9 can utilize molds made of polydimethyl siloxane (PDMS).

### Example 9

A microarray of hydrogel matrix can also be prepared in accordance with the polymerization and cross-linking steps described in Example 7. This microarray of hydrogel matrix can also be functionalized using the steps described in the Examples above.

### Example 10

The hydrogel matrix can be prepared to form a layer. The hydrogel matrix is formed from a formulation that includes small particles of a water soluble polymer such as polyvinyl alcohol or polyvinyl acetate. Once the matrix has been formed, it is washed in water or physiological saline in order to remove the diluent if any, and also dissolve out the particles. The particles leave spaces of specific volume, ranging from 10² to 10⁶ micron³, randomly and uniformly distributed into the matrix. Up to 10⁶ particles may be loaded into 1 mL of the monomer formulation. Each micro-cavity formed thereby allows access to antibody sites resident on biotin bound to avidin molecules.

### Example 11

The dendrimer (e.g., PMMA, PPI, or combination thereof) can be prepared to form a layer. The dendrimer can be formed to include functional groups. Once the matrix has been formed, it is washed in water or physiological saline in order to remove the diluent if any, and also dissolve out the particles. The particles leave spaces of specific volume, ranging from 10² to 106 micron³, randomly and uniformly distributed into the matrix. Up to 10⁶ particles may be loaded into 1 mL of the monomer formulation. Each micro-cavity formed thereby allows access to antibody sites resident on biotin bound to avidin molecules.

1. A biosensor component comprising: a substrate coated with a metal; and an anchor substance comprising a binding protein and a functional group having at least one sulfur atom, wherein the anchor substance binds directly to the metal through the functional group and forms a monolayer on the metal coated substrate; and wherein the anchor substance is configured to couple to a capture reagent.

2. The biosensor component of claim 1, wherein the metal is selected from the group consisting of aluminum, gold, and aluminum-alloy any combination thereof, or the metal is aluminum.

3. (canceled)

4. The biosensor component of claim 1, wherein the functional group is a thiol group.

5. The biosensor component of claim 1, wherein the binding protein is avidin, oligonucleotide, antibody, affimer, aptamer, or polynucleotide, or the binding protein is avidin selected from the group consisting of neutravidin, natural avidin, strepavidin, and any combination thereof.

6. (canceled)

7. The biosensor component of claim 1, wherein the capture reagent comprises a biotin moiety for binding to the binding protein of the anchor substance, or the capture reagent comprises a moiety for binding to whole cells, bacteria, eukaryotic cell, tumor cell, virus, fungus, parasite, spore, nucleic acid, small molecules or protein.

8. (canceled)

9. The biosensor component of claim 7, wherein the moiety is selected from the group consisting of antibody, affimer, or aptamer.

10. The biosensor component of claim 1, further comprising an acoustic wave transducer.

11. The biosensor component of claim 10, wherein the acoustic wave transducer generates a bulk acoustic waves (BAW) or a BAW selected from the group consisting of thickness shear mode, acoustic plate mode, and horizontal plate mode.

12. (canceled)

13. The biosensor component of claim 1, wherein the biosensor component is a film bulk acoustic-wave resonator-based (FBAR-based) device.

14. The biosensor component of claim 10, wherein the acoustic wave transducer generates a surface acoustic waves (SAW) or a SAW selected from the group consisting of shear horizontal surface acoustic wave, surface traverse wave, Rayleigh wave, and love wave.

15. (canceled)

16. The biosensor component of claim 1, wherein the substrate comprises a piezoelectric material, optionally wherein the substrate further comprises a dielectric layer and the metal is coated on the dielectric layer.

17. The biosensor component of claim 1, wherein the metal is coated directed on the substrate.

18. (canceled)

19. A bulk wave resonator comprising the biosensor component of claim 1.

20. A process of coating a surface of a metal material with a bioactive film, comprising:
applying a first composition comprising an anchor substance to the surface of the metal material to form a monolayer on the surface, wherein the anchor substance comprises a binding protein and a functional group having at least one sulfur;
applying a second composition comprising a biotinylated capture reagent to the monolayer of the anchor substance, wherein the biotinylated capture reagent binds to the anchor substance through the binding protein to form a layer of the biotinylated capture reagent.

21. The process of claim 17, further comprising subjecting the surface of the anchor substance to a plasma cleaning.

22. A biosensor component comprising:
a piezoelectric substrate;
an anchor substance bound to a surface of the piezoelectric substrate, wherein the anchor substance comprises a spacer and a binding component, and a capture reagent, wherein the anchor substance is coupled with the capture reagent thorough the binding component.

23. The biosensor component of claim 22, wherein the binding component is a binding protein, wherein the binding protein is avidin, oligonucleotide, antibody, affimer, aptamer, or polynucleotide or the binding protein is avidin selected from the group consisting of neutravidin, natural avidin, strepavidin, and any combination thereof.

24. (canceled)

25. (canceled)

26. The biosensor component of claim 22, wherein the binding component is a binding compound having one or more functional groups, wherein the binding compound has one or more functional group selected from the group consisting of N-Hydroxysuccinimide (NHS), sulfo-NHS, epoxy, carboxylic acid, carbonyl, Maleimide and amine.

27. (canceled)

28. The biosensor component of claim 22, wherein the spacer is a polymer linker selected from the group consisting of polyethylene glycol, polyvinyl alcohol, or polyacrylates.

29. (canceled)

30. (canceled)

31. The biosensor component of claim 22, wherein the anchor substance forms a layer on the surface of the piezoelectric substrate or a self-assembled monolayer on the surface of the piezoelectric substrate.

32. (canceled)

33. The biosensor component of claim 22, wherein the binding protein of the anchor substance is extended away from the surface of the piezoelectric substance through the spacer.

34. The biosensor component of claim 22, wherein the piezoelectric substrate is selected from the group consisting of quartz lithium niobate and tantalate, 36° Y quartz, 36° YX lithium tantalate, langasite, langatate, langanite, lead zirconate titanate, cadmium sulfide, berlinite, lithium iodate, lithium tetraborate, bismuth germanium oxide, Zinc oxide, aluminium nitride, and gallium nitride.

35. The biosensor component of claim 22, further comprising a housing and a fluidics chamber wherein the surface of the piezoelectric material bearing the anchor layer forma forms a wall of the chamber.

36. The biosensor component of claim 22, wherein the anchor substance binds to the surface of the piezoelectric substrate through a silane group.

37. The biosensor component of claim 22, wherein the binding protein is avidin, oligonucleotide, antibody, affimer, aptamer, or polynucleotide or wherein the binding protein is avidin selected from the group consisting of neutravidin, natural avidin, strepavidin, and any combination thereof.

38-47. (canceled)

48. A process of coating a surface of a piezoelectrical material with a biofilm, comprising:
applying a first composition comprising an anchor substance to the surface of a substrate coated with a metal to form a mono layer on the surface, wherein the anchor substance comprises a spacer coupled to a binding component; applying a second composition comprising biotinylated capture reagent to the monolayer of the anchor substance, wherein the biotinylated capture reagent binds to the anchor substance through the binding component of the anchor substance to form a layer of the biotinylated capture reagent, or
a method for determining the presence or quantity of an analyte in a sample the method comprising:
contacting the biosensor component of claim 1 with a sample;
generating an acoustic wave across the coated substrate; and measuring any change in amplitude, phase or frequency of the acoustic wave as a result of analyte binding to the capture reagent, or
a biosensor component comprising:
a piezoelectric substrate;
and
a capturing reagent immobilized on the piezoelectric substrate, wherein the piezoelectric substrate comprises a three-dimensional (3D) matrix microstructure configured to increase the number of capturing reagent immobilized on the piezoelectric substrate, and wherein the capturing reagent immobilized on the piezoelectric substrate through binding to the 3D matrix microstructure, or
a method of fabricating a biosensor component, comprising: forming a 3D matrix microstructure on a piezoelectric substrate to increase the surface area of the piezoelectric substrate; and immobilizing one or more capturing reagent on the piezoelectric substrate.

49-50. (canceled)

51. The biosensor component of claim 48, wherein the 3D matrix microstructure comprises a plurality of holes or a microarray of the capturing agents or a hydrogel matrix or a dendrimer.

52-83. (canceled)
